(19) United States
(12) Patent Application Publication
(10) Pub. No.: US 2026/0029392 A1
(43) Pub. Date: Jan. 29, 2026

(54) MICROBIAL AIR QUALITY MONITORING SYSTEM

(71) Applicant: ARIZONA BOARD OF REGENTS ON BEHALF OF ARIZONA STATE UNIVERSITY, Scottsdale, AZ (US)

(72) Inventor: Amir Elyaderani, Phoenix, AZ (US)

(21) Appl. No.: 19/283,935

(22) Filed: Jul. 29, 2025

Related U.S. Application Data
(60) Provisional application No. 63/676,563, filed on Jul. 29, 2024.

(51) Int. Cl. G01N 33/00

(52) U.S. Cl. CPC G01N 33/0075; G01N 33/0034; G01N 33/0037; G01N 33/004

(57) ABSTRACT

A device is described herein comprising a microcontroller integrated with an environmental sensor and a plurality of gas sensors. The device includes at least one application running on a processor of the microcontroller, wherein the at least one application receives environmental sensor data from the environmental sensor and the gas sensor data from the plurality of gas sensors, wherein the at least one application uses the environmental sensor data and gas sensor data to determine an environmental state of the microcontroller.

### RELATED APPLICATIONS

This application claims the benefit of U.S. Application No. 63/676,563, filed Jul. 29, 2024, the entirety of which is hereby incorporated by reference.

### TECHNICAL FIELD

The disclosure herein involves air quality monitoring systems and devices.

### BACKGROUND

Air quality monitoring is a critical field within environmental health and public safety, primarily due to its significant impact on human health and ecological balance. Traditional air quality monitoring methods have primarily relied on passive and active air sampling techniques. While these methods are effective, they are often cumbersome, expensive, and limited in their ability to provide real-time data and comprehensive microbial analysis. This limitation has become evident, for example, in high-risk environments such as healthcare facilities and food processing units, where the presence of airborne microbial contaminants can have severe consequences.

Recent technological advancements have enabled more sophisticated approaches, like laser-induced fluorescence, which can detect microbial contaminants in real time. However, these technologies are generally expensive and designed for specific high-stakes environments, making them less accessible for broader public health applications. There has been a growing need for a cost-effective, accessible, and efficient system capable of providing immediate insights into microbial air quality to facilitate quick and informed decision-making in various settings.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

In some embodiments, the present disclosure includes a device comprising, a microcontroller integrated with an environmental sensor and a plurality of gas sensors, at least one application running on a processor of the microcontroller, wherein the at least one application receives environmental sensor data from the environmental sensor and gas sensor data from the plurality of gas sensors, wherein the at least one application uses the environmental sensor data and the gas sensor data to determine an environmental state of the microcontroller.

In embodiments, the environmental sensor data comprises ambient temperature in degrees Celsius.

In embodiments, the environmental sensor data comprises relative humidity percentage.

In embodiments, the environmental sensor data comprises atmospheric pressure in Hectopascal Pressure Units.

In embodiments, the gas sensor data comprises total volatile organic compounds (VOCs) in parts per billion, wherein the total VOCs comprise ethanol, formaldehyde, benzene, and toluene initially detected in the aggregate as voltage analog reading.

In embodiments, the gas sensor data comprises estimated CO₂ levels in ppm.

In embodiments, the gas sensor data comprises general air quality data measure in parts ppm, wherein the general air quality data includes ammonia (NH₃), nitrogen oxides (NOx), alcohols, benzene, smoke, and carbon dioxide (CO₂) initially detected in the aggregate as voltage analog reading.

In embodiments, the gas sensor data comprises CO levels in parts per million.

In embodiments, the gas sensor data comprises NO₂ levels in parts per million.

In embodiments, the at least one application comprises a neural network, wherein the neural network comprises a first hidden layer, wherein the first hidden layer comprises 100 neurons, wherein the first hidden layer uses a Rectified Linear Unit (ReLU) activation function, wherein the first hidden layer is followed by a dropout layer with rate of 0.2.

In embodiments, the neural network comprises a second hidden layer, wherein the second hidden layer comprises 50 neurons, wherein the second hidden layer uses a Rectified Linear Unit (ReLU) activation function, wherein the second hidden layer is followed by a dropout layer with rate of 0.2.

In embodiments, an output layer uses comprises a single neuron using a Sigmoid activation function.

In embodiments, the environmental state comprises either an indoor environment or an outdoor environment.

In embodiments, the present disclosure discloses a microcontroller integrated with an environmental sensor and a plurality of gas sensors, at least one application running on a processor of the microcontroller, wherein the at least one application receives environmental sensor data from the environmental sensor and gas sensor data from the plurality of gas sensors, wherein the at least one application uses the environmental sensor data and the gas sensor data to determine an environmental state of the microcontroller, and the at least one application communicatively coupled with one or more applications running on one or more processors of a remote server, the at least one application transmitting the environmental sensor and gas sensor data to the one or more applications.

In embodiments, the environmental sensor data comprises environmental variables including ambient temperature in degrees Celsius, relative humidity percentage, and atmospheric pressure in Hectopascal Pressure Units.

In embodiments, the gas sensor data comprises air quality variables including total volatile organic compounds in parts per billion, estimated CO₂ levels in ppm, general air quality data measure in parts ppm, CO levels in parts per million, and NO₂ levels in parts per million.

In embodiments, the one or more applications is communicatively coupled with one or more mobile computing devices.

In embodiments, the one or more applications monitors the environmental sensor data and the gas sensor data, wherein the monitoring includes computing at least one of a moving average, an average, a max value, and a min value for at least one of the environmental variables and the at least one of the air quality variables.

In embodiments, the one or more applications generate an alert when the at least one of a moving average, an average, a max value, and a min value exceeds a given threshold value and the environmental state comprises a designated state.

In embodiments, the designated state comprises either an indoor state or an outdoor state.

### BRIEF DESCRIPTION OF THE DRAWINGS

The features and advantages of the present disclosure, and the manner of attaining them, will become more apparent and the present disclosure will be better understood by reference to the description of the present disclosure taken in conjunction with the accompanying drawings, wherein:

FIG. 1 shows an air quality monitoring device, under an embodiment.

FIG. 2 shows an air quality monitoring device, under an embodiment.

FIG. 3 shows training and validation accuracy across 100 epochs, under an embodiment.

FIG. 4 shows training and validation loss across 100 epochs, under an embodiment.

FIG. 5 shows a confusion matrix illustrating actual versus predicted labels, under an embodiment.

FIG. 6 shows a receive operating characteristic (ROC) curve, under an embodiment.

FIG. 7 shows an air quality monitoring device, under an embodiment. under an embodiment.

FIG. 8 shows an air quality monitoring device, under an embodiment.

FIG. 9 shows TensorFlow model metrics, under an embodiment.

FIG. 10 shows a visualization of the neural network, under an embodiment.

Those of ordinary skill in the art will understand that the devices, systems and methods specifically described herein and illustrated in the accompanying drawings are non-limiting exemplary embodiments and that the scope of the various embodiments of the present disclosure is defined solely by the claims. The features illustrated or described in connection with one exemplary embodiment may be combined with the features of other embodiments. Such modifications and variations are intended to be included within the scope of the present disclosure.

### DETAILED DESCRIPTION

### Example 1

An affordable monitoring system is described therein that makes air quality information more accessible, thus enabling quick action and strengthening public health defenses. The system uses cost-effective hardware like the Arduino Nano 33 BLE Sense Lite and a suite of gas sensors, coupled with software for data handling and visualization. The system is designed to be user-friendly and do-it-yourself, with a machine learning model that interprets and predicts microbial levels, offering critical insights into air quality.

Microorganisms such as bacteria, fungi, and viruses are essential to our ecosystem, populating air, water, and surfaces. These organisms emit a range of gases as metabolic byproducts. The following are exemplary gases produced by various airborne and environmental microbes including Carbon Dioxide (CO₂), Methane (CH₄), Hydrogen Sulfide (H₂S), Ammonia (NH₃), Nitrous Oxide (N₂O), Volatile Organic Compounds (VOCs), Dimethyl Sulfide (DMS), and Oxygen (O₂).

Carbon Dioxide (CO₂) is emitted by most microbes during respiration as they convert organic compounds into energy.

Methane (CH₄) is created by methanogenic archaea while decomposing organic materials under anaerobic conditions found in environments like wetlands and ruminant digestive tracts.

Hydrogen Sulfide (H₂S) is generated by sulfur-reducing bacteria, often located in oxygen-deprived settings like swamps.

Ammonia (NH₃) arises from bacterial decomposition of nitrogenous substances and the process of ammonification.

Nitrous Oxide (N₂O) is produced by nitrifying and denitrifying bacteria within the nitrogen cycle.

Volatile Organic Compounds (VOCs) include a diverse group of gases, including alcohols and hydrocarbons, is produced by bacteria and fungi. An example is geosmin, which gives soil its distinct scent.

Dimethyl Sulfide (DMS): A breakdown product of DMSP from marine phytoplankton, processed by marine bacteria.

Oxygen (O₂): A byproduct of photosynthetic microbes, not usually associated with waste but crucial for life.

These microbial gases influence the environment in various ways. Methane and nitrous oxide, for example, are significant greenhouse gases, while hydrogen sulfide is known for its toxicity and odor. VOCs, on the other hand, can lead to the formation of secondary pollutants like ozone. In environmental monitoring and bioinformatics, understanding these gas emissions is vital for evaluating ecosystem health, deciphering biogeochemical cycles, and mitigating pollution. Incorporating sensors that measure humidity, temperature, pressure, alongside gas-specific and particulate matter sensors such as the PMS7003 sensor, enhances our monitoring capabilities significantly. Examining the list of microbial gases:

Under an embodiment, CO₂ levels can indicate microbial activity. Monitoring CO₂ with environmental data helps distinguish between human and microbial emissions.

Under an embodiment, understanding methane's (CH₄) impact involves correlating its levels with temperature and pressure, as its emission varies with these factors.

Under an embodiment, H₂S solubility changes with humidity, so humidity sensors are crucial for accurate monitoring of this gas.

Under an embodiment, Ammonia's (NH₃) atmospheric dispersion can be better predicted by analyzing sensor data alongside environmental conditions.

Under an embodiment, insights into the environmental conditions favoring N₂O production can inform strategies for its control.

Under an embodiment, monitoring VOCs with environmental sensors aids in understanding conditions that lead to the formation of secondary pollutants.

Tracking DMS correlation with oceanic temperature and pressure can shed light on its role in cloud formation and climate.

Under an embodiment, elevated oxygen (O₂) levels can signal events like algal blooms, detectable through particulate matter data.

By utilizing a comprehensive suite of sensors, the system achieves a detailed understanding of the environmental conditions influencing microbial gas emissions. This integrated monitoring strategy not only aids in ecosystem health assessment but also supports the development of predictive air quality models. These models are instrumental in shaping policies and health guidelines to address the environmental and public health impacts of microbial gases.

### Virus Detection:

Viruses operate differently from bacteria and other microbes; they lack metabolic processes that produce detectable gases or compounds. As obligate intracellular parasites, they replicate exclusively within host cells, using the host's cellular machinery, thus not emitting metabolic byproducts as bacteria do.

Under an embodiment, system sensors aid in signaling the presence of viruses:

Particulate Matter Sensors: Since viruses can bind to aerosol particles, devices like the PMS7003 can sense these particles in the air. They can't pinpoint viruses, but a rise in specific-sized particulate matter can hint at viral aerosols, more so in controlled settings like hospital rooms. This requires data collection from controlled environment to be utilize for training and developing a machine learning model.

Environmental Sensors: Observing changes in temperature and humidity can offer clues about virus stability and transmission. These sensors don't detect viruses per se but can signal conditions likely to harbor or propagate viral activity.

Air Sampling: Certain sensor-triggered systems initiate air sampling when environmental thresholds, like particulate spikes, are crossed. These samples can be analyzed for viral content through PCR in labs.

Chemical Sensors: These sensors can indicate the presence of disinfectants, indicating recent cleaning that can be part of decontamination efforts against viruses.

Electronic Nose (E-Nose) Technology: Without wishing to be bound by theory, viruses can influence the host's metabolic processes, altering the VOC profile of the environment. An E-Nose can detect such shifts, offering an indirect virus detection method.

In essence, while direct viral detection is beyond the capacity of environmental sensors due to the non-metabolic nature of viruses, these sensors are valuable in monitoring conditions conducive to viral presence and aiding in subsequent air sample analysis for detailed laboratory investigation. Direct viral identification still relies on specific bioassays, molecular techniques, or advanced biosensors designed to recognize viral particles or genetic material.

### Technical Report

In exemplary embodiments of the present disclosure, a “Microbial Air Quality Monitoring System” uses various sensors paired with the Arduino Nano 33 BLE Sense to detect gases and environmental factors related to microbial presence. The Arduino Nano 33 BLE Sense, built on the nRF52840 microcontroller comprises a compact size, processing capability, BLE support, and extensive sensor compatibility.

Sensor Technical Summary: (an embodiment of the system includes the following technology and wiring configuration)

1. BME280 Environmental Sensor: This sensor provides readings of temperature, humidity, and barometric pressure, which are crucial for air quality assessment. It communicates with the Arduino via the I2C protocol.

2. Gas Sensors (CO, NO₂, NH₃): Analog sensors are used to measure carbon monoxide, nitrogen dioxide, and ammonia levels. Each one is connected to a specific analog pin on the Arduino, providing a voltage that reflects gas concentration.

3. PMS7003 Particle Sensor: Upgrading from the PMS5003, the PMS7003 detects particulate matter and communicates via a serial interface.

4. TFT Display: An Adafruit ST7789-based TFT LCD is used for real-time data visualization. It connects to the Arduino through SPI communication and several control pins.

### Wiring Details:

All sensors and the display are powered by the Arduino's 3.3V output.

The BME280's SDA and SCL lines are linked to the Arduino's I2C pins.

The gas sensors are wired directly to the analog pins and are read using the analogRead ( ) function.

The PMS7003 will be connected to the Arduino's serial pins for data transfer.

The TFT display's SPI communication lines connect to the Arduino's SPI pins, with specific GPIO pins for control as per the code.

The serial print statements in the Arduino code are crucial for debugging and tracking sensor values in real-time. Under an embodiment, data collection feeds into a machine learning model for predictive microbial air quality analytics as further described herein.

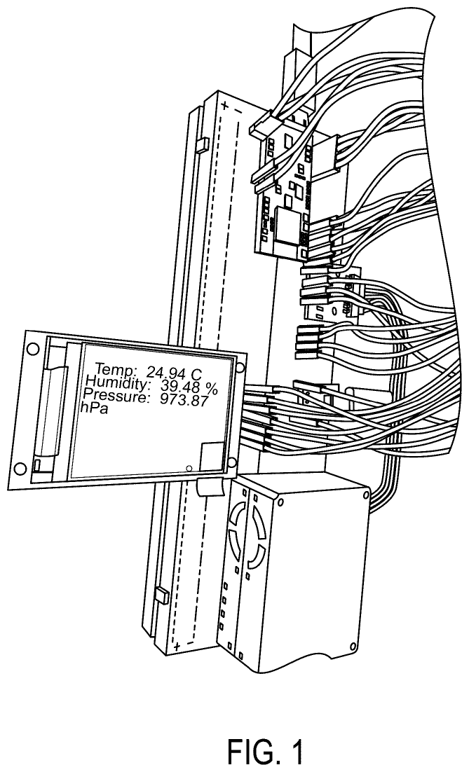

FIG. 1 shows an air quality monitoring device using PMS5003 particle module, under an embodiment.

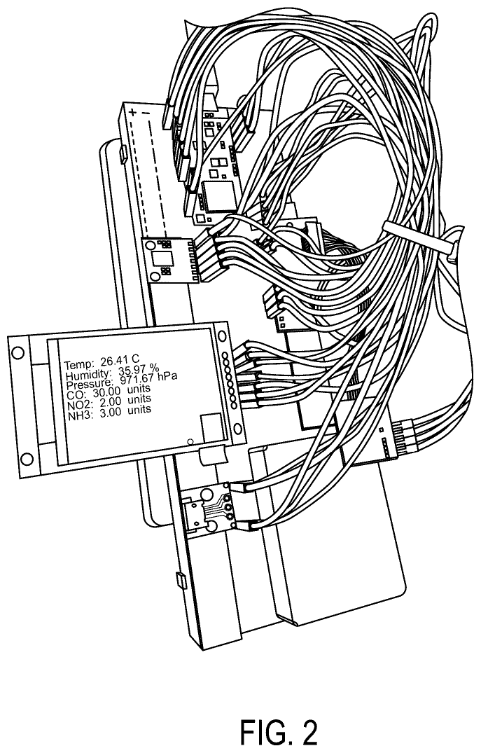

FIG. 2 shows an air quality monitoring device using a PMS7003 sensor, under an embodiment.

### Example 2

The Microbial Air Quality Monitoring System is designed to enhance the detection and analysis of airborne microbial contaminants through a novel integration of hardware and software technologies. Utilizing the compact and robust Arduino Nano 33 BLE Sense along with a suite of precise gas sensors, this system provides an affordable, user-friendly solution for real-time air quality monitoring. It incorporates advanced data handling capabilities and a machine learning model to interpret environmental conditions and predict microbial levels with enhanced precision.

A cost-effective, accessible, and reliable tool for monitoring microbial air quality is provided and which is crucial for public health, especially in environments prone to high microbial contamination like hospitals and food processing facilities. This system bridges the gap in current monitoring technologies by offering real-time analytics and reducing reliance on traditional, labor-intensive microbial detection methods.

The Microbial Air Quality Monitoring System leverages the Arduino Nano 33 BLE Sense, a compact microcontroller equipped with multiple sensor capabilities, integrated with a suite of specific gas sensors that are sensitive to microbial byproducts like CO₂, NH₃, and VOCs. This system is enhanced with machine learning algorithms that analyze sensor data to predict microbial levels, offering a significant advancement in real-time monitoring capabilities. The invention stands out by providing a user-friendly, real-time analytical tool that fills a critical gap in existing microbial air quality monitoring technologies, combining affordability with high performance and accessibility.

The Microbial Air Quality Monitoring System is designed to provide a comprehensive solution for the detection and analysis of airborne microbial contaminants using a blend of modern sensor technology and advanced data processing techniques. This system integrates hardware components for data acquisition and software components for data analysis and prediction, making it an all-inclusive tool for real-time air quality monitoring.

### Hardware Components:

Arduino Nano 33 BLE Sense: This compact and powerful microcontroller serves as the core of the monitoring system. It is equipped with built-in environmental sensors and Bluetooth Low Energy (BLE) capabilities, facilitating wireless data transmission and integration with other devices.

Gas Sensors: A suite of gas sensors, including models for detecting CO₂, NH₃, NO₂, and VOCs, are connected to the Arduino. These sensors are chosen for their sensitivity to gases commonly emitted by microbial activity. The system can be expanded with additional sensors based on specific monitoring needs.

PMS7003 Particle Sensor: An advanced particle counter that detects particulate matter sizes relevant for microbial monitoring. This sensor provides crucial data that helps in estimating microbial load in the air.

Environmental Sensors: Additional sensors like the BME280, which measures temperature, humidity, and atmospheric pressure, provide context for the gas readings, enhancing the accuracy of microbial predictions.

### Software Components:

Data Collection Software: Custom firmware written for the Arduino facilitates continuous data collection from the connected sensors. This software handles sensor initialization, data reading, and preprocessing before sending the data for analysis.

Machine Learning Model: A neural network model developed in TensorFlow runs on the microcontroller for on-device data processing. This model is trained to interpret environmental data and predict microbial levels based on historical and real-time sensor data. The model uses layers configured for high sensitivity and specificity to environmental parameters indicative of microbial presence.

Data Visualization and User Interface: Real-time data is transmitted via BLE to a user interface on a computer or mobile device. Software platforms like Blynk or ThingSpeak are used to visualize the data, allowing users to see current air quality conditions and receive alerts based on the predictive outputs of the machine learning model.

### Execution:

The system is assembled by connecting the designated sensors to the Arduino Nano 33 BLE Sense using standard electronic components like wires and breadboards for initial prototyping. Each sensor's data pin is connected to the corresponding input on the Arduino, and power supply lines are arranged to ensure stable operation. After assembly, the Arduino is programmed with the data collection software and the machine learning model.

### Usage:

Once deployed, the system begins collecting data from its environment immediately. The Arduino processes this data in real-time, utilizing the embedded machine learning model to analyze and predict microbial air quality. Users can monitor the system's outputs through the data visualization platform, which displays current conditions and trends. Alerts can be configured to notify users of undesirable changes in air quality, enabling prompt response to potential health risks.

The Microbial Air Quality Monitoring System introduces a sophisticated yet accessible approach to detecting airborne microbial contaminants, which combines advanced sensor technology with embedded machine learning capabilities. At the core of the system is the Arduino Nano 33 BLE Sense, equipped with multiple environmental sensors and enhanced by additional gas sensors sensitive to microbial byproducts such as CO₂, NH₃, and VOCs. This integration facilitates real-time data collection and analysis, enabling the system to provide timely insights into air quality conditions with a focus on microbial presence.

The invention's commercial potential is significant, for example in industries where air quality is critical to health and safety, such as healthcare, food processing, and public transportation. By offering a cost-effective, user-friendly, and efficient tool for continuous microbial monitoring, the system can help organizations comply with health regulations and improve environmental safety. Its ability to operate independently of extensive lab analyses and provide immediate data supports proactive health measures and operational decisions, potentially opening up new markets for real-time environmental monitoring solutions.

The Microbial Air Quality Monitoring System distinguishes itself from existing air quality monitoring technologies through several innovative features including, but not limited to:

Integrated Machine Learning on a Microcontroller: Unlike traditional systems that rely on external data processing, this invention integrates a neural network model directly onto the Arduino Nano 33 BLE Sense microcontroller. This allows for on-device processing of environmental data, reducing latency and eliminating the need for continuous internet connectivity or data offloading for analysis. The embedded machine learning model enables the system to predict microbial levels in real time, a capability not commonly found in conventional air quality monitoring devices.

Advanced Sensor Integration: The system utilizes a unique combination of gas sensors specifically selected for their sensitivity to microbial byproducts such as CO₂, NH₃, NO₂, and VOCs, alongside a high-performance particle sensor, the PMS7003, which provides enhanced particulate matter detection. This sensor suite is more comprehensive than those found in typical air quality monitors, allowing for more detailed and accurate assessments of microbial air quality.

Real-Time Data Visualization and Alerts: Through the integration with platforms like Blynk or ThingSpeak, the system provides real-time data visualization and alerting capabilities. Users can monitor air quality parameters through a user-friendly interface and receive immediate notifications if the air quality deteriorates beyond predetermined thresholds. This feature facilitates proactive management of environments susceptible to microbial contamination.

Cost-Effectiveness and Accessibility: The system is designed to be cost-effective and accessible, leveraging widely available components and open-source software to lower the barrier to entry for advanced air quality monitoring. This makes it appealing, for example, for applications in sectors that traditionally may not have access to sophisticated microbial monitoring tools due to cost constraints.

DIY and Modularity: The system's design encourages do-it-yourself assembly and customization, allowing users to modify or expand the system based on their specific needs. This modularity and flexibility are novel in the field of environmental monitoring, where systems are often rigid and difficult to adapt.

These innovations not only enhance the system's functionality but also expand its applicability across various industries, making it a versatile tool in the fight against air quality-related health issues.

The Microbial Air Quality Monitoring System offers several significant advantages over existing air quality monitoring technologies including, but not limited to:

Enhanced Real-Time Analysis: Traditional air quality monitors often delay data analysis due to the need to send data to external servers or laboratories. In contrast, this system integrates real-time, on-device machine learning analysis, enabling immediate identification and response to changes in air quality. This leads to faster decision-making and intervention, which is critical in environments like hospitals and food processing facilities where delays can compromise health and safety.

Comprehensive Sensor Array: By utilizing a bespoke array of sensors that detect not only conventional pollutants but also specific gases associated with microbial activity (CO₂, NH₃, NO₂, VOCs), the system provides a more comprehensive understanding of air quality. This holistic approach is a significant improvement over many systems that focus on a narrow range of contaminants.

Cost-Effectiveness: The use of affordable components like the Arduino Nano 33 BLE Sense and open-source software reduces the overall cost of the monitoring system, making it accessible to smaller facilities or organizations that previously could not afford sophisticated air quality monitoring. This democratization of technology can have a broad impact on public health, especially in under-resourced areas.

User-Friendly Interface and Data Accessibility: With real-time data visualization through platforms such as Blynk or ThingSpeak, users can easily access and interpret air quality data. The system also enables customizable alerts for when air quality deteriorates, which enhances user engagement and promotes proactive management of environmental conditions.

Flexibility and Scalability: The modular design allows users to customize or scale the system according to their specific needs. Whether expanding the number of sensors or integrating different types of sensors, the system can adapt to a wide range of environmental conditions and user requirements, unlike many rigid, proprietary systems.

Environmental Impact: By enabling more organizations and individuals to monitor and respond to air quality issues effectively, the system contributes to environmental health and can significantly reduce the exposure to harmful microbial contaminants in various settings. These advantages position the Microbial Air Quality Monitoring System as a leading solution in environmental monitoring, capable of meeting the dynamic needs of modern public health and safety standards while pushing the boundaries of what is possible in microbial air quality assessment.

The Microbial Air Quality Monitoring System is positioned to transform the landscape of environmental health monitoring with its advanced, cost-effective technology tailored for real-time microbial contamination detection. This system's innovative integration of affordable sensor technology and embedded machine learning makes it a potent candidate for commercialization in several lucrative markets.

### Exemplary Products and Services:

Healthcare Facilities Monitoring Solutions: Customizable systems for hospitals and clinics to continuously monitor air quality and manage infection risks, especially in high-risk areas like operating rooms and ICUs.

Food Industry Compliance: Systems designed for use in food processing and storage facilities to ensure compliance with health and safety standards by monitoring microbial levels that can affect product quality and safety.

HVAC Integrated Systems: Integrated solutions for HVAC systems in commercial and residential buildings to enhance air quality management and energy efficiency through smarter air handling decisions based on real-time data.

Portable Devices for Individual Use: Compact, user-friendly devices for personal monitoring in homes or in workplaces, for example, beneficial for individuals with health conditions that make them sensitive to air quality.

Smart City Infrastructure: Integration into urban environmental monitoring systems to improve public health reporting and pollution control, providing city administrators and public health officials with real-time data on air quality trends and anomalies.

Traditional Air Quality Monitoring Systems: While there are numerous air quality monitoring systems available on the market, many focus primarily on chemical pollutants rather than microbial contaminants. Traditional systems also tend to rely on lab-based analysis or are prohibitively expensive for continuous monitoring.

Real-Time PCR and Other Lab Techniques: High-precision but high-cost technologies such as real-time PCR offer detailed insights into microbial presence but lack the real-time monitoring capabilities and require specialized personnel and equipment.

IoT-Enabled Environmental Sensors: Various IoT solutions offer real-time data; however, they often require cloud connectivity for data processing and do not integrate machine learning at the hardware level, which can delay response times and increase operational costs. The Microbial Air Quality Monitoring System's ability to provide immediate, actionable insights directly from the device without needing external data processing or specialized infrastructure presents a competitive edge. This system not only fills a gap in current technology by offering real-time monitoring of both chemical and microbial air quality but also does so in a cost-effective manner that democratizes access to advanced air quality monitoring.

Regulatory Compliance: Increasingly stringent regulations on air quality and public health safety in various industries drive demand for more rigorous monitoring solutions. Public Health Awareness: Greater public awareness of the health impacts of poor indoor air quality, for example post-pandemic, has increased demand for monitoring solutions, especially those that can detect microbial contaminants.

Technological Advancements: Advances in sensor technology and machine learning provide new opportunities for developing more accurate and responsive air quality monitoring systems. By leveraging these market dynamics, the Microbial Air Quality Monitoring System is well-positioned to capture significant market share and meet the growing demand for sophisticated yet accessible air quality monitoring solutions.

### Example 3

A novel Microbial Air Quality Monitoring System is described herein that detects airborne microbial contaminants. Utilizing the Arduino Nano 33 BLE Sense and a suite of gas sensors, the system offers an affordable, user-friendly solution for real-time air quality monitoring. It integrates data handling and a machine learning model to interpret environmental conditions, aiming to predict microbial levels. The system's performance has been advanced by incorporating sophisticated particle sensors and additional gas sensors. Challenges include obtaining necessary approvals from health organizations.

Air quality, a vital aspect of public health and environmental science, has garnered increased attention due to its impact on human health and ecological balance. Traditional methods for monitoring air contaminants are often cumbersome, expensive, and inaccessible for real-time data analysis. This project addresses these limitations by developing a Microbial Air Quality Monitoring System, designed to provide an innovative, cost-effective, and user-friendly solution for the detection and analysis of airborne microbial contaminants.

Our novel contribution lies at least in the integration of the Arduino Nano 33 BLE Sense with an array of specific gas sensors—each selected for their sensitivity to microbial byproducts such as CO2, NH3, and VOCs. The system employs machine learning algorithms to analyze the sensor data, enabling the system to predict microbial levels with greater precision than traditional methods. This approach offers a significant advancement in real-time monitoring capabilities, empowering users to respond promptly to changing air quality conditions.

The system's innovation extends to its design, which seamlessly combines hardware with software analytics. By leveraging embedded machine learning, the system facilitates on-device data processing, negating the need for constant connectivity and data offloading for analysis. This integrated approach ensures that our system is not only effective in laboratory settings but also practical for deployment in varied environments—from urban infrastructure to remote locations—thereby filling a critical gap in current monitoring technologies.

In the realm of environmental monitoring, for example, within the context of public health and manufacturing safety, microbial air quality monitoring stands as a critical discipline. Traditional methods for assessing airborne microbial contamination have primarily relied on passive and active air sampling technologies. Passive monitoring typically involves the use of ‘settle plates,’ which are Petri dishes containing non-selective culture media exposed to the environment to capture viable biological particles that sediment from the air. Although inexpensive and straightforward, these methods offer only qualitative data and are limited in scope, missing smaller particles that remain suspended in the air, thus failing to provide a comprehensive representation of air quality.

Active air sampling represents a more advanced approach, requiring devices that draw a known volume of air through a particle collection device. Among these, impingers and impactors are predominant. Impingers collect particles in a liquid medium and can provide quantitative results but can damage microbial cells, affecting viability. In contrast, impactors employ a solid or adhesive medium, such as agar, providing convenience and the ability to handle larger sample volumes necessary for environments like cleanrooms where microbial presence is minimal.

Despite these advancements, the existing methods are not without limitations. For instance, impaction samplers do not support the rapid enumeration and characterization of microorganisms, necessitating reliance on conventional culture methods, which are time-consuming. Moreover, the mechanical stress of the sampling process can affect microbial viability, and special care must be taken to avoid medium desiccation.

Recently, technological innovations have introduced instruments capable of real-time detection of airborne microorganisms through laser-induced fluorescence. Such devices mark a significant step forward, providing immediate detection and enumeration of microbial contaminants. However, these systems are generally designed for high-stakes environments like pharmaceuticals and may not be as applicable or affordable for broader public health applications.

In this context, our system seeks to bridge the gap by integrating real-time data processing with a unique algorithmic approach to microbial air quality monitoring. Unlike the conventional methodologies, our system employs a suite of gas sensors and environmental parameters, interpreted by a machine learning model, to infer microbial contamination levels. This novel approach not only promises to offer immediate insights into air quality but also circumvents the need for culture-based methods, accelerating the response to potential microbial hazards.

The introduction of our system also expands the scope of application to a wider range of environments, potentially transforming air quality monitoring practices by providing a cost-effective, user-friendly solution. Our project's commitment to utilizing accessible hardware like the Arduino Nano 33 BLE Sense, coupled with sophisticated software for real-time analytics, positions it as an innovative contribution to the field of microbial air quality monitoring.

### Exemplary System Design

### Technical Design Overview

Our Microbial Air Quality Monitoring System is designed as a multi-sensor integration project, combining the compact and robust Arduino Nano 33 BLE Sense with a series of gas sensors to detect and quantify environmental conditions indicative of microbial presence. By leveraging the computational efficiency of embedded systems with the analytic power of machine learning, the system establishes a predictive model that not only monitors but also anticipates changes in air quality due to microbial activities.

### Sensor Selection and Data Acquisition

The system uses selected a range of sensors for their sensitivity and specificity to the gases commonly associated with microbial metabolism, including:
BME280 Environmental Sensor: Measures ambient temperature, humidity, and pressure, providing context for the gas sensor readings.
MQ Series Gas Sensors: A suite of sensors that detect CO, NO2, NH3, and other gases related to microbial presence.
PMS7003 Particle Sensor: An upgrade from the PMS5003, it offers enhanced detection of particulate matter, a potential carrier of microbes.

These sensors are interfaced with the Arduino Nano 33 BLE Sense through a combination of I2C and analog connections. Data from each sensor is read at predetermined intervals, capturing a snapshot of the environmental conditions.

### System Architecture

The system architecture is bifurcated into hardware data acquisition and software-based data analysis. The hardware layer is centered around the Arduino Nano 33 BLE Sense, which functions as the central data hub. The software layer encompasses data preprocessing, feature extraction, and machine learning model execution, running primarily on the Arduino's onboard microcontroller with TensorFlow Lite for microcontrollers.

### Model Architecture:

The neural network model architecture for the Microbial Air Quality Monitoring System is a well-designed structure, tailored to process and interpret the multi-dimensional dataset from various environmental sensors. Its input layer is specifically adapted to handle this complex dataset, with the layer's size influenced by the distinct features from the sensor readings.

The network includes two hidden layers, each crucial in the model's learning process. The first hidden layer has 100 neurons and uses the ReLU (Rectified Linear Unit) activation function, enabling the model to capture non-linear relationships in the data. A dropout layer follows, with a 0.2 rate, to prevent overfitting and ensure a more generalizable outcome. The second hidden layer consists of 50 neurons with the ReLU activation function, enhancing the model's learning. It's followed by another dropout layer with the same rate, enhancing the model's generalization ability. The output layer has a single neuron with a sigmoid activation function, ideal for binary classification tasks, like distinguishing between indoor and outdoor conditions. The model's compilation uses the Adam optimizer for its efficiency with sparse gradients and adaptability. It employs the binary cross-entropy loss function, standard for binary classification, with accuracy as the key metric during training and evaluation.

### Evaluation Approach:

In the “Microbial Air Quality Monitoring System” project, focusing on indoor and outdoor environment prediction, our evaluation approach was designed to ensure a thorough and accurate assessment of the system's performance. This included a detailed method for data collection, followed by the application of specific evaluation metrics.

### Data Collection Methods:

Environment and Duration: Data collection was conducted in various indoor and outdoor environments, including urban and rural areas, homes, offices, and public buildings. The collection period lasted several weeks to capture different environmental conditions over time.

Conditions of Data Collection: The Arduino Nano 33 BLE Sense, with gas sensors, was used for data collection. These sensors measured environmental parameters like temperature, humidity, and gas concentrations (CO, NO₂, NH₃, particulate matter). Data was labeled as ‘indoor’ or ‘outdoor’ for the supervised learning approach.

Data Handling: A Python script processed the data from multiple CSV files into a comprehensive dataset. The data was cleaned, normalized, and divided into training and testing sets for the machine learning phase.

### Evaluation Metrics:

In evaluating the “Microbial Air Quality Monitoring System,” key metrics were used to assess the system's effectiveness and efficiency. Accuracy is a primary metric, measuring the proportion of correctly classified instances in the dataset. High accuracy is crucial for the system's reliability, especially in real-world applications of air quality monitoring.

Alongside accuracy, the F-score is important in the evaluation. It provides a balanced view of the model's precision and recall, essential in scenarios with significant consequences from false positives and negatives. Evaluating the F-score ensures an understanding of the model's predictive capabilities, balancing the detection of actual events against false alarms. Model size is another important factor. With the system designed for embedded devices, optimizing model size is crucial. A smaller model size allows faster inference times and lower power consumption, vital for sustainable, continuous monitoring.

Real-time performance, focusing on the model's ability to promptly analyze data, is also crucial. This includes assessing the latency between data acquisition and prediction output, ensuring effective operation in real-time scenarios, essential for air quality monitoring.

In summary, the evaluation approach for this subproject was well-crafted, including a thorough data collection process and the application of stringent evaluation metrics. These metrics, chosen to assess the model's accuracy, efficiency, practicality, and applicability in real-time and varied settings, highlight the system's readiness for deployment in air quality monitoring.

### Results:

In the subproject of the “Microbial Air Quality Monitoring System,” focusing on predicting indoor and outdoor environments with sensor data, the results from the neural network model evaluation are described herein. These findings are crucial in understanding our approach's effectiveness in real-world scenarios.

Training and Validation Performance: During training, the model consistently improved in learning the dataset's characteristics. It achieved a high accuracy, with a training accuracy of 80.67% and a validation accuracy of 95.98%. This high validation accuracy indicates the model's robustness and generalization ability.

Test Performance: On the test dataset, the model showed a high accuracy of 95.98%. This consistent performance across training, validation, and testing underlines the model's reliability and effectiveness in accurately classifying environmental conditions.

Comparison with Existing Techniques: Our system, integrating specific gas sensors with a machine learning model, shows improvement in real-time analysis and effectiveness on an embedded system, though direct comparisons with existing techniques are limited.

Model Size and Power Consumption: The optimized TensorFlow Lite model, suitable for deployment on the Arduino Nano 33 BLE Sense, is compact, allowing for quick inference and low power consumption, essential for field operation. This optimization maintained the model's accuracy, making it a viable solution for low-power, real-time monitoring.

Optimization Impacts: Converting to a TensorFlow Lite model involved quantization and other techniques, reducing the model size with minimal accuracy impact, demonstrating the effectiveness of these strategies for deployable machine learning solutions on embedded devices.

Handling Unbalanced Data: Techniques like stratified sampling and data augmentation addressed dataset imbalances. A balanced training-validation split ensured exposure to a mix of indoor and outdoor environments, reducing prediction bias.

In conclusion, our subproject results show the potential of integrating environmental sensors with machine learning for real-time air quality monitoring. The model's high accuracy, optimized size, and efficient power consumption make it a promising tool for environmental health and safety applications. Its ability to handle unbalanced data enhances its applicability in diverse scenarios.

### Example 4

Airborne microbial contamination poses a significant risk in various settings like healthcare facilities, food processing units, and residential spaces. Traditional microbial monitoring systems are often expensive and complex to operate.

Ensuring a safe and healthy environment by monitoring and controlling microbial air quality is crucial. A simplified, cost-effective monitoring system can democratize access to critical air quality information, aiding in timely intervention and improved public health.

Under an embodiment, a microbial monitoring system includes the following software and hardware components:

### Hardware Components:

Microcontroller: Arduino Nano 33 BLE Sense Lite—a compact, affordable microcontroller with built-in environmental sensors.
Particle Counter: Plantower PMS7003—operates on laser scattering principle to estimate particulate matter, serving as a proxy for microbial load.
Additional Environmental Sensors: Optional sensors like BME280 for enhanced environmental monitoring.
Data Logging: Micro SD card module for local data storage.
Power Supply: A suitable power adapter or battery pack.

### Software Components:

Data Collection Software: Custom Arduino sketch to continuously collect and log data from sensors.
Machine Learning Model: A predictive model trained on historical microbial data (if available) or synthetic data to estimate microbial concentrations.
   Today our model is a NN model that has been trained and tested on indoor and outdoor data from the user
Data Visualization Tool: Platforms like Blynk or ThingSpeak for real-time data visualization.

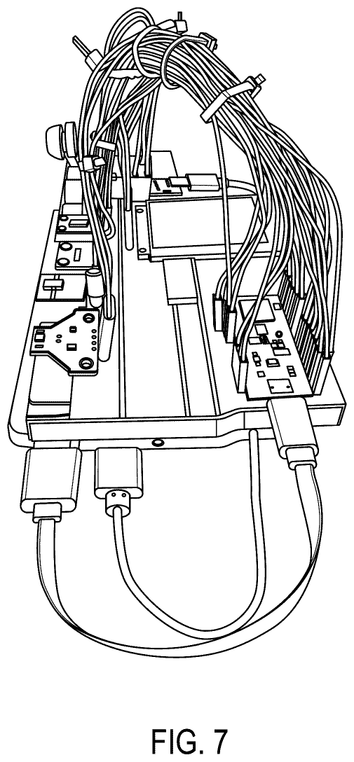

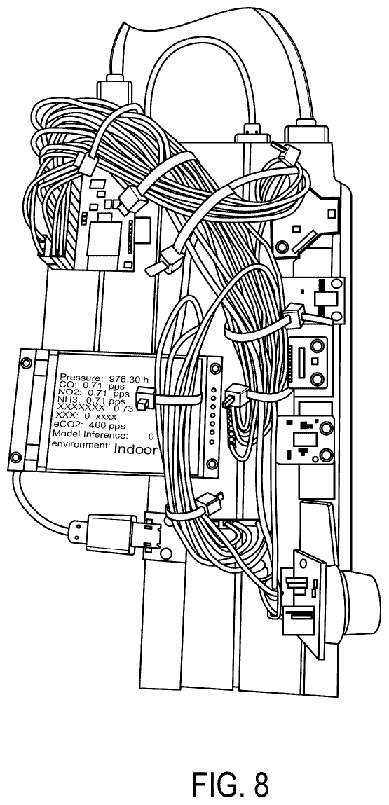

A machine learning model is designed and implemented to classify environmental conditions using sensor data, under an embodiment. Key Objectives include collecting and labeling sensor data with Arduino Nano 33 BLE Sense, analyzing data for pattern detection, and training a neural network for indoor vs. outdoor classification. Outcomes include developing a model with high accuracy, supported by detailed metrics. and enabling real-time prediction of environmental settings. The system integrates hardware data acquisition with data analytics and deploys a machine learning algorithm in an embedded system for direct inference. See FIGS. 7 and 8 for an example of the system device, under an embodiment.

Under an embodiment, the microcontroller includes the following sensor which detect corresponding environmental parameters as described herein:

### BME280 Environmental Sensor:

Temperature: Ambient temperature measurement in ° C.
Humidity: Relative humidity percentage.
Pressure: Atmospheric pressure in hPa.

### SGP30 Gas Sensor:

TVOC: Total Volatile Organic Compounds in ppb (Ethanol, Formaldehyde, Benzene, and Toluene (initially detected in the aggregate as voltage analog reading)
eCO₂: Estimated CO₂ levels in ppm.

### MQ-135 Gas Sensor:

General air quality detection including various gases and smoke (Ammonia (NH₃), Nitrogen Oxides (NOx), Alcohols, Benzene, Smoke, and Carbon Dioxide (CO₂) initially detected in the aggregate as voltage analog reading).

### MiCS-5524 Gas Sensor:

Detection of CO, NO₂, and other gases, output as ppm Under an embodiment, an overview of the model includes the following:

### Libraries & Frameworks:

TensorFlow for model building and training, NumPy for numerical computations, Pandas for data manipulation, Matplotlib & Seaborn for visualization, Scikit-learn for performance metrics.

### Data Handling:

Preprocessing function placeholder ready for customization. Best model tracking by accuracy and seed value. EarlyStopping with monitor=′val_loss′, min_delta=0.001, patience=10.

### Model Training:

Iterative training over 100 seeds for reproducibility. CSV data loading and splitting: 80% training, 20% testing. TensorFlow Dataset with batching (size 30) and prefetching.

### Model Architecture:

Sequential model with layers: Dense (100 neurons, ReLU activation). Dropout (0.2 rate). Dense (50 neurons, ReLU activation). Dropout (0.2 rate). Dense (1 neuron, Sigmoid activation).
Trained for up to 100 epochs with early stopping on validation data.

### Overview

The machine learning model utilized in the Microbial Air Quality Monitoring System is designed to predict environmental conditions, specifically distinguishing between indoor and outdoor settings based on sensor data. This model is implemented using TensorFlow and Keras, and it is trained on a dataset comprising various environmental parameters collected by sensors integrated with the Arduino Nano 33 BLE Sense microcontroller.

### Model Architecture

The neural network model is constructed as a Sequential model in Keras, consisting of the following layers:

### 1. Input Layer:

The input layer is configured to handle multiple features derived from the sensor data. Each feature represents a specific environmental parameter such as temperature, humidity, gas concentrations (e.g., CO₂, NH₃, VOCs), and particulate matter levels.

### 2. First Hidden Layer:

Dense Layer: This layer contains 100 neurons and uses the Rectified Linear Unit (ReLU) activation function. The choice of 100 neurons allows the model to capture a wide range of patterns and relationships within the data.
Dropout Layer: A dropout rate of 0.2 is applied to this layer to prevent overfitting. This means that 20% of the neurons are randomly ignored during each training iteration, which helps in generalizing the model to unseen data.

### 3. Second Hidden Layer:

Dense Layer: This layer comprises 50 neurons, also using the ReLU activation function. The reduced number of neurons compared to the first hidden layer helps in progressively refining the feature space.
Dropout Layer: Another dropout layer with a 0.2 rate is included to further prevent overfitting.

### 4. Output Layer:

Dense Layer: The output layer consists of a single neuron with a Sigmoid activation function. This configuration is ideal for binary classification tasks, outputting a probability value between 0 and 1, indicating whether the environment is classified as indoor or outdoor.

### Model Compilation

The model is compiled with the following configurations:
Optimizer: Adam optimizer is used due to its efficiency in handling sparse gradients and its adaptability. It helps in faster convergence and better performance.
Loss Function: Binary cross-entropy loss is employed as it is well-suited for binary classification problems. This function calculates the difference between the predicted probabilities and the actual class labels.
Metrics: Accuracy is the primary metric used to evaluate the model's performance during training and validation phases.

### Data Handling and Preprocessing

The sensor data is preprocessed to ensure it is suitable for training the neural network. Key steps include:
Data Cleaning: Removing any anomalies or missing values from the dataset.
Normalization: Scaling the sensor readings to a uniform range, typically between 0 and 1, to facilitate effective learning by the model.
Data Splitting: The dataset is divided into training (80%) and testing (20%) sets to evaluate the model's performance.

### Training and Evaluation

The training process involves the following steps:
Epochs: The model is trained for up to 100 epochs, with early stopping implemented to halt training if the validation loss does not improve for 10 consecutive epochs. This helps in preventing overfitting and saves computational resources.
Batch Size: The data is processed in batches of 30 samples, which balances the trade-off between computational efficiency and model performance.

### Performance Metrics

The model's performance is evaluated using several metrics:
Accuracy: Measures the proportion of correctly classified samples.
Confusion Matrix: Provides a detailed breakdown of true positives, true negatives, false positives, and false negatives, helping to understand the model's classification performance.
ROC Curve: The Receiver Operating Characteristic (ROC) curve and the Area Under the Curve (AUC) provide insights into the model's ability to distinguish between classes. An AUC close to 1 indicates excellent performance.

### Model Performance

The model achieves high accuracy in distinguishing between indoor and outdoor environments, with training accuracy reaching 80.67% and validation accuracy at 95.98%. The ROC curve demonstrates an AUC of 0.98, indicating strong predictive capabilities. The confusion matrix shows a high number of correct predictions with minimal misclassifications.

### CONCLUSION

The neural network model developed for the Microbial Air Quality Monitoring System effectively leverages sensor data to predict environmental conditions with high accuracy. The combination of robust preprocessing, a well-designed neural network architecture, and comprehensive evaluation metrics ensures that the model is reliable and suitable for real-time air quality monitoring applications.

The Microbial Air Quality Monitoring System employs an advanced neural network model designed to predict environmental conditions by classifying them as either indoor or outdoor settings. This model is implemented using the TensorFlow and Keras libraries, and it is trained on a diverse dataset of environmental parameters gathered through various sensors integrated with the Arduino Nano 33 BLE Sense microcontroller. The architecture of this neural network model is meticulously crafted to handle the complexity and variability of the input data, ensuring accurate and reliable predictions.

The model begins with an input layer configured to process multiple features derived from sensor data, including temperature, humidity, gas concentrations such as CO₂, NH₃, VOCs, and particulate matter levels. These features provide a comprehensive snapshot of the environmental conditions, which are crucial for distinguishing between indoor and outdoor environments. The input layer feeds into the first hidden layer, which consists of 100 neurons.

Under an embodiment, the model utilizes the following input variables to predict indoor or outdoor environmental states:
relative humidity percentage
atmospheric pressure in Hectopascal Pressure Units
total volatile organic compounds in parts per billion
estimated CO₂ levels in ppm
general air quality data measured in parts per million
CO in parts per million
NO₂ in parts per million

Each of these input variables is preprocessed using min-max normalization to scale their values between 0 and 1, ensuring uniform contribution to the model. The resulting normalized values are then compiled into structured input vectors and fed into the first hidden layer of the neural network, which consists of 100 neurons. This layer uses the Rectified Linear Unit (ReLU) activation function, a popular choice in deep learning due to its ability to handle non-linear relationships within the data efficiently. Additionally, a dropout rate of 0.2 is applied to this layer to mitigate the risk of overfitting, where 20% of the neurons are randomly ignored during each training iteration. This dropout mechanism ensures that the model generalizes well to unseen data by preventing it from becoming too tailored to the training dataset.

Following the first hidden layer is a second hidden layer with 50 neurons, also utilizing the ReLU activation function. The reduction in the number of neurons from the first to the second hidden layer helps in refining the feature space and focusing the model's learning capacity. Another dropout layer with a rate of 0.2 is applied here, further enhancing the model's ability to generalize. The final layer of the model is the output layer, which comprises a single neuron with a Sigmoid activation function. This configuration is ideal for binary classification tasks, producing a probability value between 0 and 1 that indicates whether the environment is classified as indoor or outdoor.

The model is compiled using the Adam optimizer, known for its efficiency in handling sparse gradients and adaptability, facilitating faster convergence and improved performance. The binary cross-entropy loss function is employed, as it is well-suited for binary classification problems by quantifying the difference between the predicted probabilities and the actual class labels. Accuracy is chosen as the primary metric to evaluate the model's performance during both the training and validation phases.

Preprocessing the sensor data is a critical step to ensure it is suitable for training the neural network. This process involves cleaning the data to remove anomalies or missing values, normalizing the sensor readings to a uniform range, typically between 0 and 1, and splitting the dataset into training (80%) and testing (20%) sets. The normalization step is important as it allows the neural network to process the data more effectively, ensuring that each feature contributes equally to the model's learning process.

Training the model involves iterating through the dataset in batches, with each epoch representing a complete pass through the training data. The model is trained for up to 100 epochs, with early stopping implemented to prevent overfitting. Early stopping halts the training process if the validation loss does not improve for 10 consecutive epochs, thereby saving computational resources and preventing the model from becoming overly complex. The batch size is set to 30 samples, balancing computational efficiency and model performance.

Evaluating the model's performance is conducted using several key metrics. Accuracy measures the proportion of correctly classified samples, providing a straightforward indication of the model's effectiveness. The confusion matrix offers a detailed breakdown of true positives, true negatives, false positives, and false negatives, allowing for a more nuanced understanding of the model's classification performance. Additionally, the Receiver Operating Characteristic (ROC) curve and the Area Under the Curve (AUC) provide insights into the model's ability to distinguish between classes. An AUC close to 1 indicates excellent performance, demonstrating the model's strong predictive capabilities. The model has achieved high accuracy in distinguishing between indoor and outdoor environments, with training accuracy reaching 80.67% and validation accuracy at 95.98%. The ROC curve demonstrates an AUC of 0.98, further highlighting the model's proficiency. The confusion matrix shows a high number of correct predictions with minimal misclassifications, underscoring the model's reliability. This high level of performance is maintained despite the challenges of balancing the dataset and optimizing the model for deployment on an embedded system like the Arduino Nano 33 BLE Sense.

In conclusion, the neural network model developed for the Microbial Air Quality Monitoring System leverages advanced machine learning techniques to provide accurate real-time predictions of environmental conditions. The combination of robust preprocessing, a well-designed neural network architecture, and comprehensive evaluation metrics ensures that the model is not only effective but also practical for real-time air quality monitoring applications. The detailed implementation and optimization steps underscore the model's readiness for deployment, offering a significant advancement in the field of environmental health monitoring.

Under an embodiment, the Microbial Air Quality Monitoring System (i.e., the Arduino Nano 33 BLE Sense) is communicatively coupled with one or more applications running on a remote server. The application(s) running on the Arduino microcontroller monitor air quality parameters in an environment. These parameters include one or more of relative humidity percentage, atmospheric pressure in Hectopascal Pressure Units, total volatile organic compounds in parts per billion, estimated CO₂ levels in ppm, general air quality data measure in parts ppm, CO in parts per million, and NO₂ in parts per million.

The one or more remote server applications are further communicatively coupled (through general internet or other network connectivity) with remote computing devices. The Arduino application(s) transmit monitored parameter values to the remote server applications. In turn the remote server application(s) send this real time data to remote computing platforms which may display parameter values in real time. (Alternatively the Arduino board is directly connected to an electronic display for real time presentation of monitored data). Either the Arduino or remote server applications (or both) may generate an alert if any one of the monitored parameters exceeds a certain threshold. The alert may be pushed out to the remote computing devices.

As indicated above, the machine learning model utilized in the Microbial Air Quality Monitoring System (and implemented by the Arduino application(s)) is designed to predict environmental conditions, specifically predicting the presence of the Arduino board in an indoor or outdoor state based on sensor data. As the model runs, the system may also log (for each monitored parameter) a moving average of parameter measurements over a period of time prior the model's prediction of an indoor or outdoor state. As opposed to a moving average, the system may log a standard average of the parameter measurements or max/min value (as applicable to the given parameter) over the same period of time. Under an embodiment, the Arduino application(s) and/or the remote server application(s) only provide an alert if (i) the system predicts a given state and (ii) the moving average/average/or max/min value of one or more parameters exceeds or falls below a given threshold.

Under an embodiment, an Arduino Portenta H7 microcontroller is used. This microcontroller provides significantly expanded memory and processing capabilities compared to the Arduino Nano 33 BLE Sense, thereby enabling the integration and concurrent execution of multiple machine learning models.

This embodiment coordinates at least two distinct models running on the device:
1. A visual inference model that uses data from a fisheye lens camera to approximate the spatial geometry of the room, including width, depth, and ceiling height.
2. A second model that processes gas concentration measurements within that space, trained on controlled environments with known volumes and contamination levels, to infer microbial density or safety thresholds.

The Arduino Portenta H7 includes a dual-core architecture and significantly more RAM, making it suitable for high-throughput applications and embedded multi-model inference. The Portenta H7 enables embedding additional models trained on diverse sensor datasets. These models collaboratively assess complex environmental states, enabling more nuanced predictions of microbial safety or contamination.

This upgraded hardware platform supports future scalability, allowing the system to evolve into a robust framework for real-time, edge-based microbial air quality analytics.

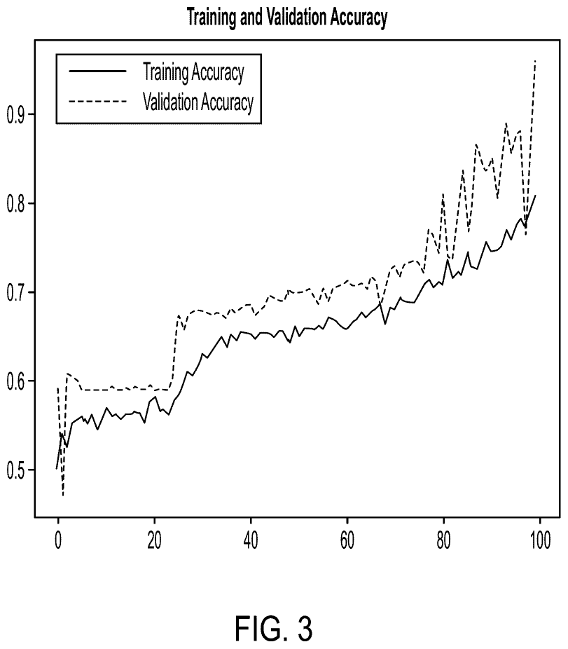

FIG. 3 shows training and validation accuracy across 100 epochs, under an embodiment.

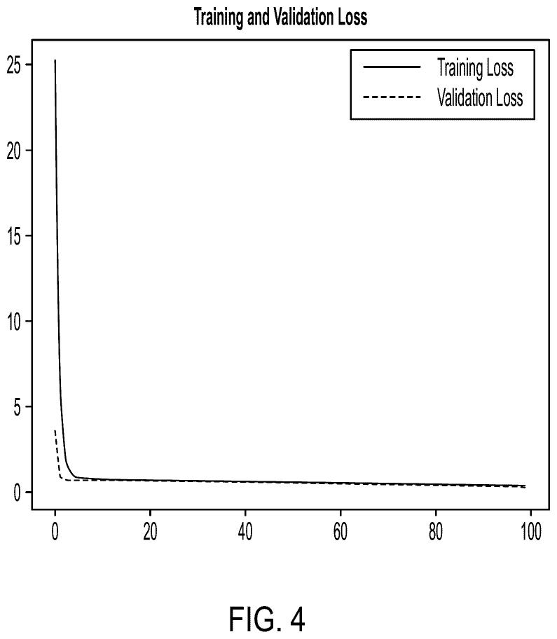

FIG. 4 shows training and validation loss across 100 epochs, under an embodiment.

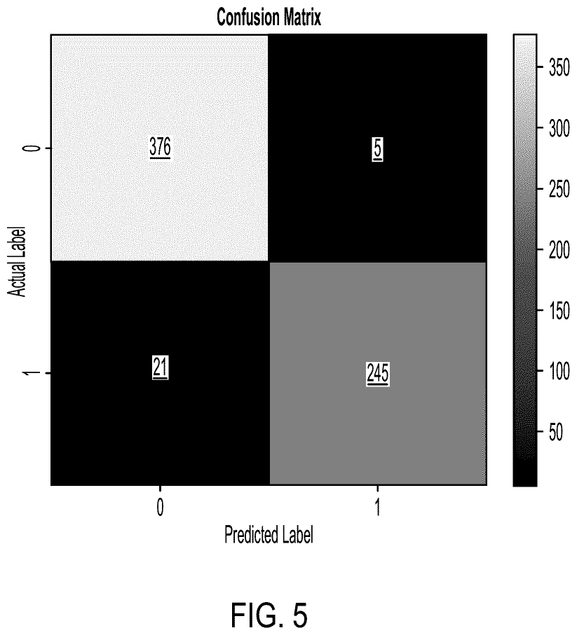

FIG. 5 shows a confusion matrix illustrating actual versus predicted labels, under an embodiment.

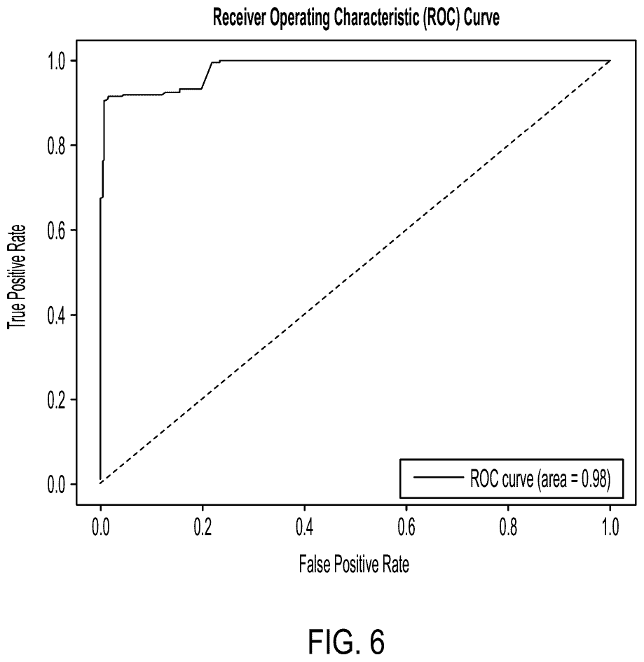

FIG. 6 shows a receive operating characteristic (ROC) curve, under an embodiment.

FIG. 7 shows an air quality monitoring device using a PMS7003 sensor, under an embodiment.

FIG. 8 shows an air quality monitoring device using a PMS7003 sensor, under an embodiment.

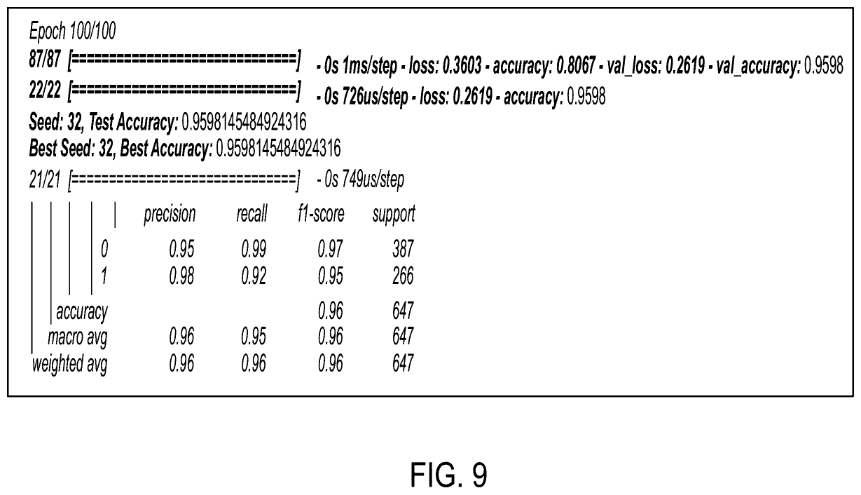

FIG. 9 shows TensorFlow model metrics, under an embodiment.

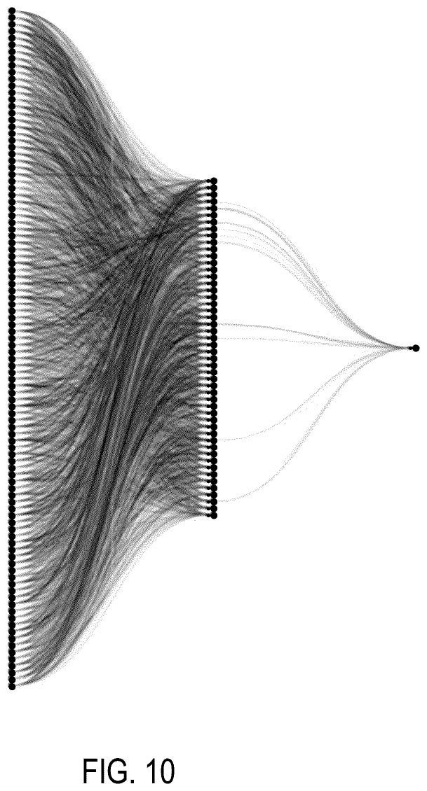

FIG. 10 shows a visualization of the neural network, under an embodiment.

Under an embodiment, the system is designed to predict whether the microcontroller is located in an indoor or outdoor environment based on the collected sensor data. Under another embodiment, the system detects the number of individuals in a room using the same parameters. The model is specific to one room and its volume. A camera module calculates room dimensions and volume to generate enough training data to develop a model that predicts the number of people in any room based on inferences from a secondary model that predicts room dimensions and volume.

Under an embodiment, the system is designed to predict whether the microcontroller is located in an indoor or outdoor environment based on the collected sensor data. Under another embodiment, the system detects the number of individuals in a room using the same parameters. The model is specific to one room and its volume.

The classification system is designed to categorize the number of individuals in a room into two classes:

Class 0: Represents environments with fewer than 5 individuals.

Class 1: Represents environments with 5 or more individuals.

This binary classification helps in determining crowd density, which can be critical for applications such as ventilation control, resource allocation, and emergency response.

The framework of the model remains largely the same as the one described in the application, with a few enhancements to accommodate the additional functionality of detecting the number of individuals in a room. The model comprises:

### Model Architecture:

Input Layer: Handles multiple features derived from sensor data, including temperature, humidity, gas concentrations (e.g., CO2, NH3, VOCs), and particulate matter levels.

First Hidden Layer: Contains 100 neurons using the Rectified Linear Unit (ReLU) activation function. This layer captures a wide range of patterns and relationships within the data.

Dropout Layer: A dropout rate of 0.2 is applied to prevent overfitting, ensuring the model generalizes well to unseen data.

Second Hidden Layer: Comprises 50 neurons, also using the ReLU activation function, further refining the feature space.

Dropout Layer: Another dropout layer with a 0.2 rate is included to enhance the model's generalization ability.

Output Layer for Environmental Classification: A single neuron with a Sigmoid activation function, ideal for binary classification tasks (indoor vs. outdoor).

Additional Output Layer for Crowd Density Classification: Another single neuron with a Sigmoid activation function, specifically for classifying crowd density (less than 5 individuals vs. 5 or more individuals).

### Model Compilation:

Optimizer: Adam optimizer is used due to its efficiency in handling sparse gradients and adaptability, facilitating faster convergence and better performance.

Loss Function: Binary cross-entropy loss is employed for both classification tasks (environmental classification and crowd density classification). This function calculates the difference between the predicted probabilities and the actual class labels.

Metrics: Accuracy is the primary metric used to evaluate the model's performance during training and validation phases.

### Data Handling and Preprocessing:

Data Cleaning: Removing any anomalies or missing values from the dataset.

Normalization: Scaling the sensor readings to a uniform range (typically between 0 and 1) to facilitate effective learning by the model.

Data Splitting: Dividing the dataset into training (80%) and testing (20%) sets to evaluate the model's performance.

### Training and Evaluation:

Epochs: The model is trained for up to 100 epochs, with early stopping implemented to halt training if the validation loss does not improve for 10 consecutive epochs, preventing overfitting.

Batch Size: The data is processed in batches of 30 samples, balancing computational efficiency and model performance.

### Performance Metrics:

Accuracy: Measures the proportion of correctly classified samples.

Confusion Matrix: Provides a detailed breakdown of true positives, true negatives, false positives, and false negatives.

ROC Curve: The Receiver Operating Characteristic (ROC) curve and the Area Under the Curve (AUC) offer insights into the model's ability to distinguish between classes.

These modifications ensure that the model not only predicts whether the environment is indoor or outdoor but also estimates crowd density, making it more versatile and useful for real-world applications.

Under yet another embodiment, a camera module calculates room dimensions and volume to generate enough training data to develop a model that predicts the number of people in any room based on inferences from a secondary model that predicts room dimensions and volume.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network can be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network can include one of more LANs, WANs, Internets, and computers. The computers can serve as servers, clients, or a combination thereof.

The microbial air quality monitoring system can be a component of a single system, multiple systems, and/or geographically separate systems. The microbial air quality monitoring system can also be a subcomponent or subsystem of a single system, multiple systems, and/or geographically separate systems. The components of microbial air quality monitoring system can be coupled to one or more other components of a host system or a system coupled to the host system.

One or more components of the microbial air quality monitoring system and/or a corresponding interface, system or application to which the microbial air quality monitoring system is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the microbial air quality monitoring system can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the microbial air quality monitoring system and corresponding systems and methods described herein can be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the microbial air quality monitoring system and corresponding systems and methods include: microcontrollers with memory (such as electronically erasable programmable read only memory (EEPROM)), embedded microprocessors, firmware, software, etc. Furthermore, aspects of the microbial air quality monitoring system and corresponding systems and methods can be embodied in microprocessors having software-based circuit emulation, discrete logic (sequential and combinatorial), custom devices, fuzzy (neural) logic, quantum devices, and hybrids of any of the device types described herein. Of course the underlying device technologies can be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein can be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions can be embodied include, but are not limited to, non-volatile storage media in various forms (e.g., optical, magnetic or semiconductor storage media) and carrier waves that can be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the components described herein can be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The description herein of embodiments of the microbial air quality monitoring system is not intended to be exhaustive or to limit the systems and methods to the precise forms disclosed. While specific embodiments of, and examples for, the microbial air quality monitoring system and corresponding systems and methods are described herein for illustrative purposes, various equivalent modifications are possible within the scope of the systems and methods, as those skilled in the relevant art will recognize. The teachings of the microbial air quality monitoring system and corresponding systems and methods provided herein can be applied to other systems and methods, not only for the systems and methods described herein.

The elements and acts of the various embodiments described herein can be combined to provide further embodiments. These and other changes can be made to the microbial air quality monitoring system and corresponding systems and methods in light of the above detailed description.

1. A device comprising,
a microcontroller integrated with an environmental sensor and a plurality of gas sensors;
at least one application running on a processor of the microcontroller, wherein the at least one application receives environmental sensor data from the environmental sensor and gas sensor data from the plurality of gas sensors, wherein the at least one application uses the environmental sensor data and the gas sensor data to determine an environmental state of the microcontroller.

2. The device of claim 1, wherein the environmental sensor data comprises ambient temperature in degrees Celsius.

3. The device of claim 1, wherein the environmental sensor data comprises relative humidity percentage.

4. The device of claim 1, wherein the environmental sensor data comprises atmospheric pressure in Hectopascal Pressure Units.

5. The device of claim 1, wherein the gas sensor data comprises total volatile organic compounds (VOCs) in parts per billion, wherein the total VOCs comprise ethanol, formaldehyde, benzene, and toluene initially detected in the aggregate as voltage analog reading.

6. The device of claim 1, wherein the gas sensor data comprises estimated CO₂ levels in ppm.

7. The device of claim 1, wherein the gas sensor data comprises general air quality data measure in parts ppm, wherein the general air quality data includes ammonia (NH₃), nitrogen oxides (NOx), alcohols, benzene, smoke, and carbon dioxide (CO₂) initially detected in the aggregate as voltage analog reading.

8. The device of claim 1, wherein the gas sensor data comprises CO levels in parts per million.

9. The device of claim 1, wherein the gas sensor data comprises NO₂ levels in parts per million.

10. The device of claim 1, wherein the at least one application comprises a neural network, wherein the neural network comprises a first hidden layer, wherein the first hidden layer comprises 100 neurons, wherein the first hidden layer uses a Rectified Linear Unit (ReLU) activation function, wherein the first hidden layer is followed by a dropout layer with rate of 0.2.

11. The device of claim 10, wherein the neural network comprises a second hidden layer, wherein the second hidden layer comprises 50 neurons, wherein the second hidden layer uses a Rectified Linear Unit (ReLU) activation function, wherein the second hidden layer is followed by a dropout layer with rate of 0.2.

12. The device of claim 11, wherein an output layer uses comprises a single neuron using a Sigmoid activation function.

13. The device of claim 1, wherein the environmental state comprises either an indoor environment or an outdoor environment.

14. A system comprising,
a microcontroller integrated with an environmental sensor and a plurality of gas sensors;
at least one application running on a processor of the microcontroller, wherein the at least one application receives environmental sensor data from the environmental sensor and gas sensor data from the plurality of gas sensors, wherein the at least one application uses the environmental sensor data and the gas sensor data to determine an environmental state of the microcontroller;
the at least one application communicatively coupled with one or more applications running on one or more processors of a remote server, the at least one application transmitting the environmental sensor and gas sensor data to the one or more applications.

15. The system of claim 14, wherein the environmental sensor data comprises environmental variables including ambient temperature in degrees Celsius, relative humidity percentage, and atmospheric pressure in Hectopascal Pressure Units.

16. The system of claim 15, wherein the gas sensor data comprises air quality variables including total volatile organic compounds in parts per billion, estimated CO₂ levels in ppm, general air quality data measure in parts ppm, CO levels in parts per million, and NO₂ levels in parts per million.

17. The system of claim 16, wherein the one or more applications is communicatively coupled with one or more mobile computing devices.

18. The system of claim 17, wherein the one or more applications monitors the environmental sensor data and the gas sensor data, wherein the monitoring includes computing at least one of a moving average, an average, a max value, and a min value for at least one of the environmental variables and the at least one of the air quality variables.

19. The system of claim 18, wherein the one or more applications generate an alert when the at least one of a moving average, an average, a max value, and a min value exceeds a given threshold value and the environmental state comprises a designated state.

20. The system of claim 19, wherein the designated state comprises either an indoor state or an outdoor state.
